(19) United States
(12) Patent Application Publication
(10) Pub. No.: US 2011/0223112 A1
(43) Pub. Date: Sep. 15, 2011

(54) UNSATURATED POLYESTER COATED MAGNETIC ULTRA-FINE PARTICLES FOR BIOLOGICAL APPLICATIONS

(75) Inventors: Mahmoudi Morteza, Tehran (IR); Mohammad Imani, Tehran (IR); Abdolreza Simchi, Tehran (IR)

(73) Assignee: IRAN POLYMER AND PETROCHEMICAL INSTITUTE, Tehran (IR)

(21) Appl. No.: 12/723,646

(22) Filed: Mar. 14, 2010

(51) Int. Cl. A61K 49/18; A61P 35/00

(52) U.S. Cl. 424/9.323

(57) ABSTRACT

Unsaturated polyesters e.g. poly (ethylene glycol fumarate) (PEGF) were developed as new coating materials for iron oxide nanoparticles. Different strategies were adopted in their synthesis to provide different characteristics including solubility, molecular weight and structure also degrees of unsaturation. After synthesis of the nanoparticles; the material was applied as a coating on them. These materials were applicable without further processing, however, coatings were cured via thermal, redox or photo initiated crosslinking on the nanoparticles to provide rigid shells on the surface of nanoparticles.

### FIELD OF THE INVENTION

The present invention relates to ultra fine particles applicable in molecular and cellular tracking and/or imaging, medical imaging, drug delivery or simultaneous imaging and drug delivery. More particularly, the present invention relates to method of application of an unsaturated polymeric coating on the surface of magnetic particles and crosslinking of this polymeric shell to a rigid and stable hydrogel coating upon a chemical reaction. The obtained material is a promising candidate in surface modification to carry ligands for (simultaneous) imaging and/or targeted drug delivery purpose.

### GENERAL BACKGROUND OF THE INVENTION

The applicability of magnetic particles looks very promising in drug delivery and targeting, molecular and cellular tracking and sorting and contrast enhancement in magnetic resonance imaging however; there are three major shortcomings associated with application of these materials in magnetically targeted drug delivery, imaging or cellular/molecular tracking. These limitations are as follows:
Toxicity of magnetic particles (such as superparamagnetic iron oxide nanoparticles) especially Maghemite which dictates application of a coating as shell on the particles.
Common coatings are based on Dextran, PVA, PEG, PEI, polyacrylic acid, PLGA, chitosan and pullulan adsorbed on the magnetic ultra fine particles surface by weak Van der Waals forces, hence the applied coating on the particles are not stable in long term and will be washed out and leave the particles bare. Once the surface-derivatized fine particles were inside the cells, it will be probable that cells may digest the polymeric coatings, leaving the bare particles exposed to other components and organdies within the cytoplasm, which could the influence the overall integrity of the cells.
The previously described coatings are of limited usefulness in drug delivery and targeting. Coating of drug and/or homing devices for targeting on the particles surface will increase the risks associated with the possibility of faster drug release (i.e. burst effect). Therefore; there will be very low amounts (if no) of drug for delivery after reaching to the affected site. Conjugation of drug molecules to the polymer coatings is reported by some researchers to overcome this problem but to obtain the designed biological effects these bonds should break at the target site which is difficult to be predicted and controlled. The obtained material in this invention could overcome these problems.

### DESCRIPTION

### Background

Due to their ultra-fine size, biocompatibility and different useful magnetic (such as superparamagnetic) properties, magnetic particles are emerging as promising candidates for various biomedical applications such as enhanced resolution magnetic resonance imaging, drug delivery, tissue repair, cell and tissue targeting and transfection, molecular and cellular tracking etc. magnetic particles with a mean particle diameter of about 10 nm suspended in appropriate carrier liquids are commonly called ferrofluids and have outstanding properties. These particles contain only a single magnetic domain and can thus be treated as small, thermally agitated magnets in the carrier liquid. The special feature of ferrofluids is the combination of normal liquid behavior with superparamagnetic properties. This enables the use of magnetic forces for the control of properties and flow of the liquids, giving rise to numerous technical applications. For instance, during in vivo applications, such as drug delivery, superparamagnetism is an activation mechanism because once the external magnetic field is removed, the magnetization disappears, and thus the agglomeration, and hence the possible embolization of the capillary vessels can be avoided.

A minimally invasive approach, in which a fluid containing magnetic nanoparticies (magnetic fluid) is injected directly into superficial or deep-seated tumors, was developed for interstitial thermotherapy. In vitro studies have shown the excellent power absorption characteristics of magnetic fluids in an alternating magnetic field. The feasibility and efficacy of magnetic ultrafine particle thermotherapy has been demonstrated in preclinical studies.

there major shortcomings encountered in application of these particles In vivo include their destabilization due to the adsorption of plasma proteins and the non-specific uptake by the reticulum-endothelial system (RES) together with their biocompatibility which relate to synthesis method and type of coating. Due to high specific surface area of these nano-sized particles, plasma proteins interact with the particles which can cause an increase in the particle size and often results in agglomeration. The particles are also considered as an intruder by the innate immune systems and can be readily recognized and engulfed by macrophage cells that may cause agglomeration. In both cases, the particles will be removed from the blood circulation which will yield a decrease in their effectiveness, leading to a reduction in efficiency of nanoparticle-based diagnostics and therapeutics. To inhibit both phenomena and provide longer circulation times, the particles are usually coated with hydrophilic and biocompatible polymers/molecules such as polyethylene glycol (PEG), dextran, polyvinyl alcohol (PVA), polyacrylic acid, poly (lactide-co-glycolide) (PLGA), chitosan, pullulan, poly (ethyleneimine) (PEI). Furthermore, the high burst effect which is related to the high surface-to-volume ratio of nanoparticles (note that drug will be loaded on the surface of nanoparticles) caused the achievement of little amount of drug to the targeted site.

Among a couple of possible strategies to achieve an in situ hydrogel formation system, using unsaturated polyesters seems as the suitable alternative candidates due to their potential ability to form cross-linked networks via their unsaturated double bonds. Then, by using a photo-curing unit or any other safe chemical method the injected materials can be easily cured. Fumaric acid containing macromers are highly unsaturated and can be cross-linked with or without using a cross-linking agent to form their corresponding polymeric networks. Currently, a number of cross-linking agents are being used in these systems because they can enhance the polymerization efficiency while imparting specific properties to the network.

The aim of the present invention is to use cross-linked poly (ethylene glycol)-co-fumarate (PEGF) as a shell in order to increase biocompatibility, stability (due to the hydrogel formation) of nanoparticles as well as decrease the burst effect considering loaded drug.

### SUMMARY

Unsaturated polyesters e.g. poly (ethylene glycol fumarate) (PEGF) were developed as new coating materials for iron oxide nanoparticles. Different strategies were adopted in their synthesis to provide different characteristics including solubility, molecular weight and structure also degrees of unsaturation. After synthesis of the nanoparticles; the material was applied as a coating on them. These materials were applicable without further processing, however, coatings were cured via thermal, redox or photo initiated crosslinking on the nanoparticles to provide rigid shells on the surface of nanoparticles.

The combination of magnetic nanoparticles coated with unsaturated shell provided different behaviors regarding thickness, water absorption and hydrophilic-hydrophobic properties and improved biocompatibility profile. The coated ultrafine particles provided high capacity for trapping of bioactive agents inside within their shell structure where showed high potential to decrease the burst release phenomenon and can be used for both drug delivery and imaging purposes.

Digestion of these polymers was delayed inside the cells and according to our results; the compositions based on these unsaturated aliphatic polyesters are potentially useful to develop novel carriers for cellular/molecular tracking, drug delivery and imaging applications or a combination of thereof.

### DESCRIPTION OF THE DRAWINGS

FIG. 1: FTIR spectra of PEGF (uncured and cured)

FIG. 2: ¹HNMR spectra of PEGF (uncured)

FIG. 3: TEM images of (a) and (b) illustrated the SPION. (c) Diffraction pattern of magnetite nanoparticles shown spinel structure

FIG. 4: SEM images of coated SPION (a) and (b) with different stirring rates and molarities

FIG. 5: Magnetization curves for (a) MNP (magnetic nanoparticles), (b) PEGF-MNP (non-cross linked coated SPION), and (c) C-PEGF-MNP (cross linked coated SPION).

FIG. 6: FT-IR spectrum of PEGF coated SPION before and after crosslinking

FIG. 7: SEM images of (a), (b) and (c) PEGF crosslinked magnetic beads

FIG. 8: (a) MTT assay results for C-PEGF-MNP (cross linked coated SPION) sample on (a) L929 and (b) K562 cells over 24 and 48 h.

FIG. 9: Release profile of PEGF-MNP and C-PEGF-MNP

### EXAMPLE 1

### PEGF Crosslinked Coating on the Surface of Iron Oxide Nanoparticles

### Detailed Description

Iron chloride and sodium hydroxide (NaOH) of analytical grades were supplied by Merck Inc. (Darmstadt, Germany) and used without further purification. PEG diol (Mw=1 kDa), fumaryl chloride (FuCl), calcium hydride and propylene oxide (PO) were all purchased from Aldrich (Milwaukee, Minn., USA). Sodium hydroxide (NaOH), ammonium per-sulfate and methylene chloride (DCM) were obtained from Merck (Germany). FuCl was purified by distillation at 161° C. under ambient pressure. Anhydrous DCM was obtained by distillation under reflux condition for 1 hr in the presence of calcium hydride. Other solvents were reagent grades and used without any further purification.

### Synthesis of PEGF

There are some methods for preparation of PEGF reported in literature. In this work PEGF macromers were synthesized according to the procedure illustrated in Scheme I. Typically, 0.03 mole of PEG diol was dissolved in 100 ml of anhydrous methylene chloride (DCM) in a three-necked 250 ml reaction flask equipped with a reflux condenser and a magnetic stirrer. Propylene oxide (PO) was added to the mixture in a 2:1 molar ratio. The purified FuCl was dissolved in 50 ml of the same solvent and added drop wise in 1 hr to the stirred reaction flask at −2° C. under nitrogen atmosphere. The reaction temperature was then raised to the room temperature and run overnight. Upon completion of the reaction, the product was washed several times with 0.1N NaOH to extract the resulted byproducts such as chlorinated propanol. The PEGF macromer was then obtained by rotovaporation, dried at 25° C. in vacuum for 24 hrs, and then stored at −15° C. until further use.

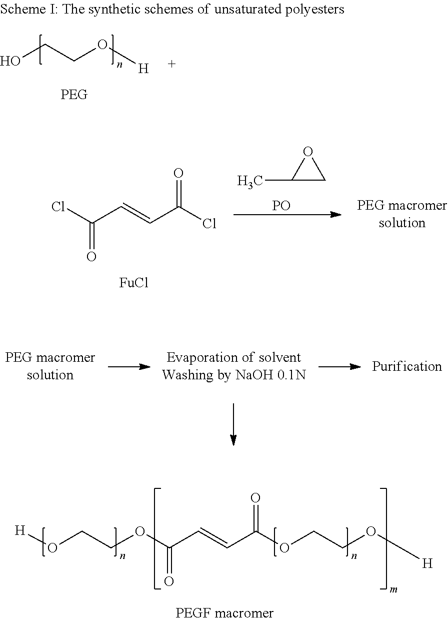

### Synthesis of Super Paramagnetic Iron Oxide Nanoparticles Coated by PEGF

Solutions were prepared using deionized (DI) water after 30 minutes bubbling with argon for deoxygenation. The iron salts were dissolved in DI water containing 1M HCL where the mole fraction of Fe²⁺ to Fe³⁺ was adjusted to 2:1 for all samples. The precipitation was performed by drop wise addition of iron salt solutions to NaOH solutions under an argon atmosphere. In order to control mass transfer, which may allow particles to combine and build larger polycrystalline particles, turbulent flow was created by placing the reaction flask in an ultrasonic bath and changing the homogenization rates (in the first 2 minutes of the reaction). Two molarities of the NaOH solution where also examined. After 30 min (with homogenization at suitable rate), the solution was centrifuged and re-dispersed in DI water several times. Then PEGF was added by syringe to the solution in the appropriate stirring rate and remained for 1 hour in order to coat the surface of SPION.

The particles were collected by centrifugation and re-dispersed in DI water. Finally, unsaturated polyesters coating were cross-linked by redox polymerization in the presence of chemical initiators. For example here ammonium persulphate had used as initiator system¹⁹ and an optimized amount of accelerator (DMAEMA) were added to the mixture and mixed thoroughly for suitable time. An interesting result was that even in the ultrahigh centrifugation rate for several minutes, no precipitation formed. It may due to the formation of hydrogel on the surface of SPION and decreased their density. As a result high stable ferrofluid suspend achieved. The obtained ferrofluid was kept at 4° C. for future usage.

### Characterization

The synthesized nanoparticles were characterized as follows. Morphology and size of the particles was investigated by TEM (ZEISS, EM-10C, and Germany) operating at 100 kV and SEM (Philips-XL30). To prepare samples for TEM, a drop of the suspension was placed on a copper grid and dried. Fourier transform infrared (FTIR) spectra (4000-400 cm⁻¹) were obtained on a Broker, Equinox 55 spectrophotometer at 4 cm⁻¹ resolution and 32 scans. All samples were prepared as KBr discs. ¹HNMR spectra were recorded in CDCl₃ at 25° C. (Broker Ultrashield® 400 MHz, Germany) and chemical shifts were recorded in ppm. Phase characterization was accomplished using XRD (Siemens, D5000, and Germany) technique with Cu Kα radiation and Schemer method for particle size determination. XRD samples were prepared by drying the obtained particles in a vacuum oven at 40° C. for 12 h after centrifugation. The magnetization of the samples in a variable magnetic field was measured using a vibrating sample magnetometer (VSM) with a sensitivity of 10⁻³ emu and magnetic field up to 20 kOe. The magnetic field was changed uniformly with a time rate of 66 Oe/s.

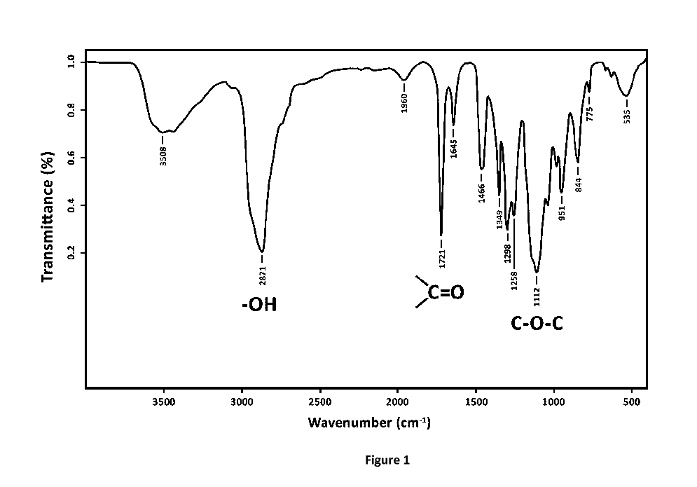

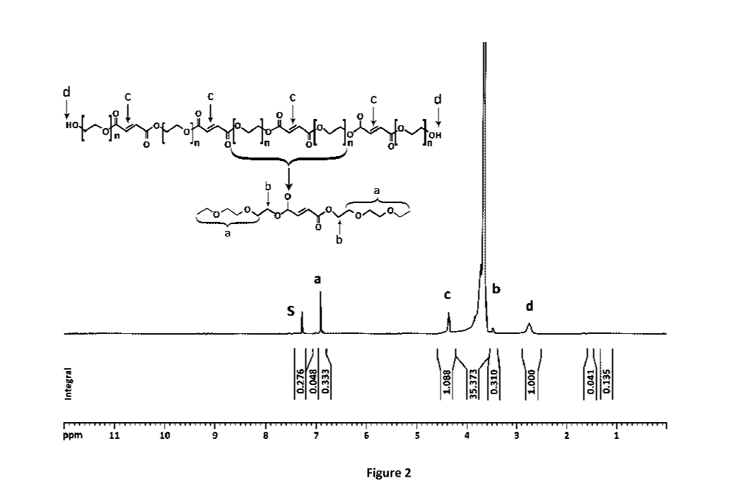

The FTIR spectra of 1 kDa PEGF are presented in FIG. 1. Asymmetrical C—O—C stretching band at 1100 cm⁻¹, C═C stretching at 1645 cm⁻¹, carbonyl stretching at 1720 cm⁻¹, strong methylene absorption at 2871 cm¹, methylene scissoring and asymmetric bending at 1455 cm¹, and hydroxyl absorption at 3442 cm⁻¹ are evident and can be found. The absorption bands presented at 950 and 858 cm⁻¹ positioned in the FTIR spectra are characteristic of the crystalline phase of PEG.

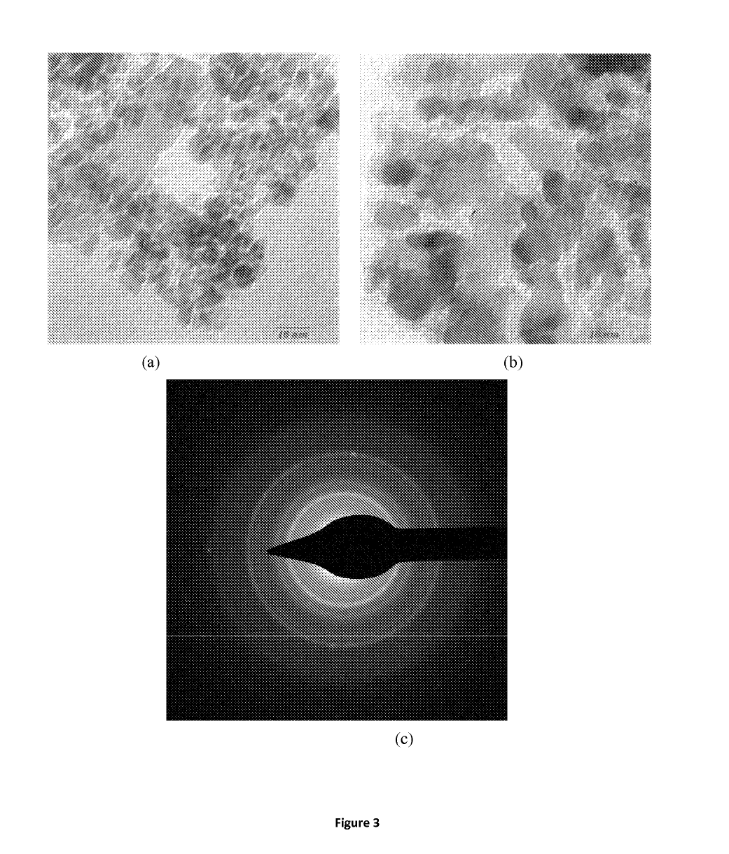

¹HNMR spectra of the synthesized PEGF macromers are shown in FIG. 2. The chemical shifts with peak positions at 3.63, 4.33, 2.7, and 6.8 ppm are due to the protons of PEG main ethylene (b), methylene groups adjacent to the fumarate groups (c), the hydroxyl group of PEG (d), and hydrogens of the fumarate group (a), respectively. Since the chemical shift of the fumarate hydrogens is below 7.0 ppm, the steric configuration of the fumarate functional groups in the copolymer should be in the cis position. The presence of chemical shift at 6.8 ppm clearly indicates that fumarate groups are incorporated in PEG.

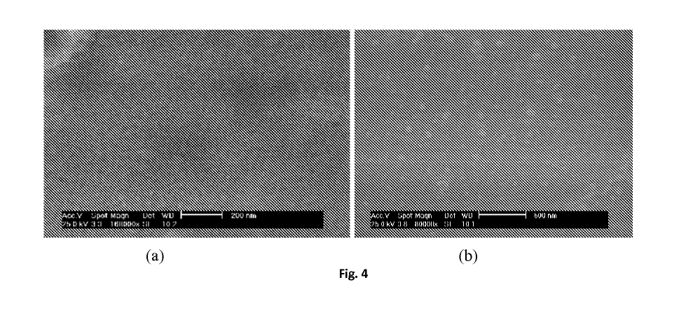

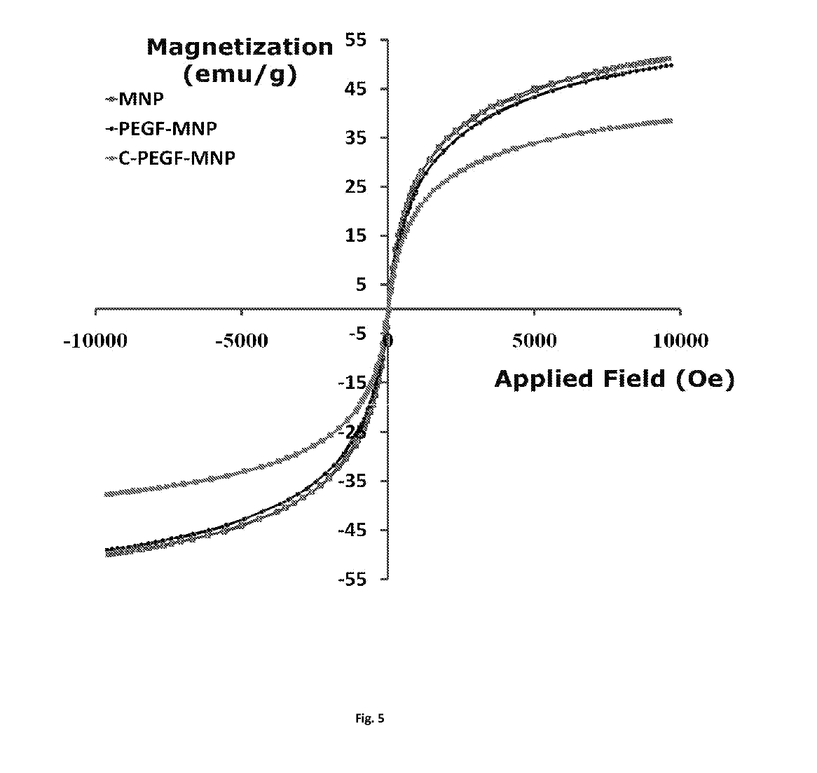

Transmission electron microscopy (TEM) of magnetite nanoparticles reveals spherically-shaped iron oxide nanoparticles (FIG. 3 (a)). Based on the TEM results SPION with narrow size distribution have been achieved. More specifically, for a given stirring rate and molarity, it appears that fixing the stirring rate and increasing the molarity favors the formation of spherical and bigger magnetic beads (nanoparticles dispersed in polymeric substrate (FIG. 3 (b)). The diffraction pattern of magnetite nanoparticles is shown in FIG. 3 (c). In order to support this idea, scanning electron microscopy (SEM) is used. FIG. 4 illustrate ultra fine nanoparticles coated with PEGF.

The samples were analyzed by VSM and showed superparamagnetic behavior with different magnetic saturations. FIG. 5 illustrate hysteresis loops of the synthesized nanoparticles showing a negligible remanence and coercivity in the hysteresis loops. If the size of magnetite will be bigger that 27-30 nm, the superparamagnetic behavior will be vanished. As a result, the particles on SEM results are not single particles (they are magnetic beads: e.g. random dispersion of nanoparticles in polymeric beads). Several researchers have reported that the magnetic saturation of superparamagnetic magnetite increases when the size of the magnetite increases, which can be attributed to the increase of weight and volume of magnetite nanoparticles. According to these studies, the magnetic properties can be lower than that of the bulk phase which is 88 emu/g.

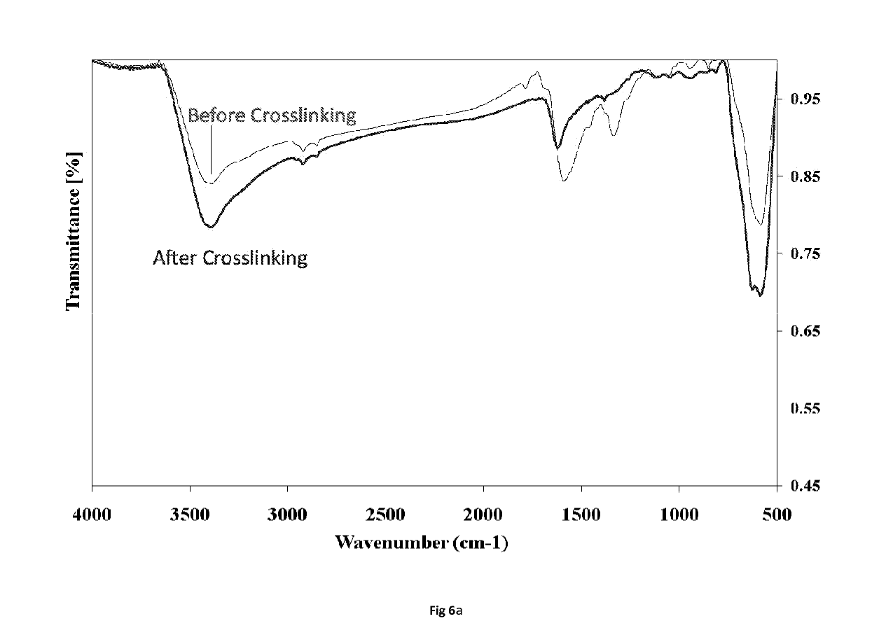

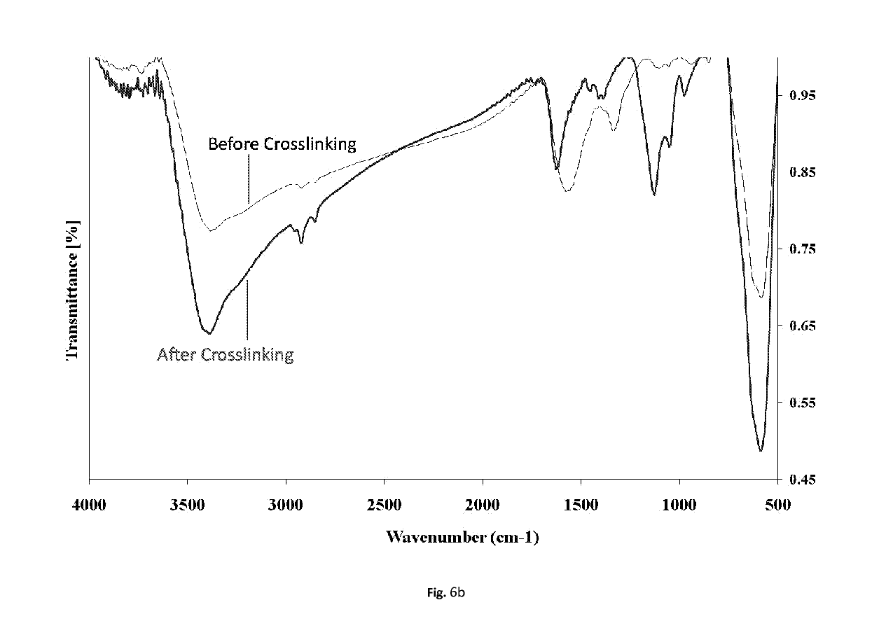

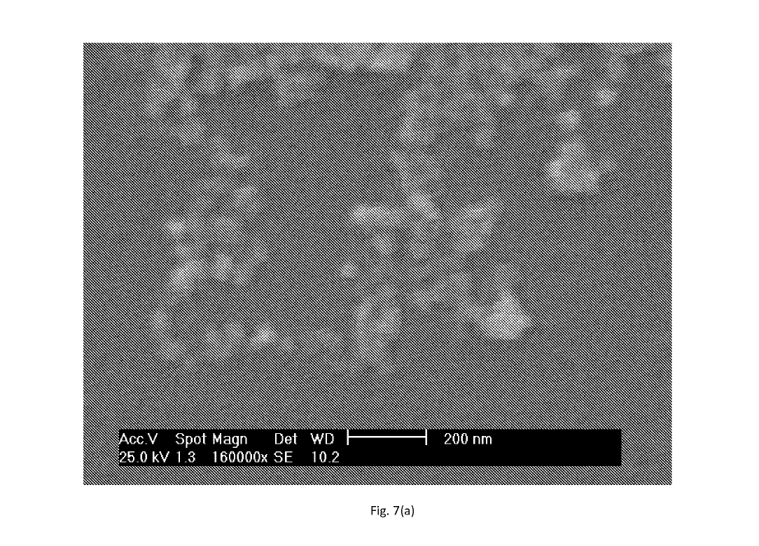

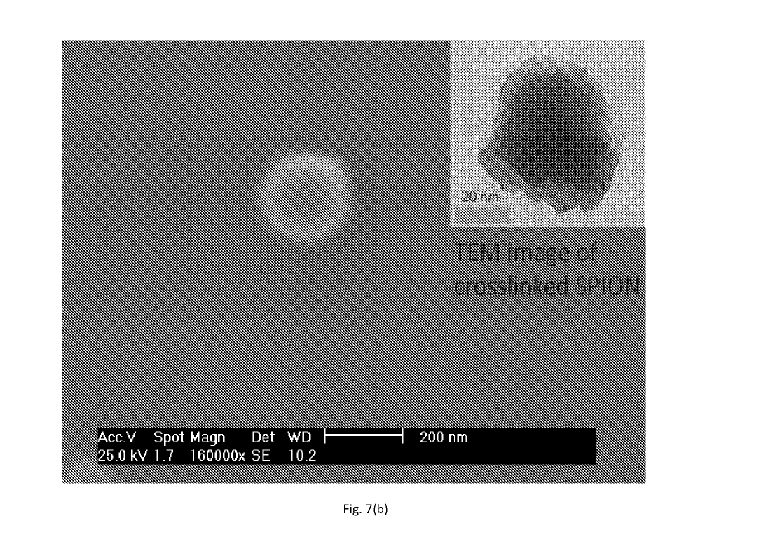

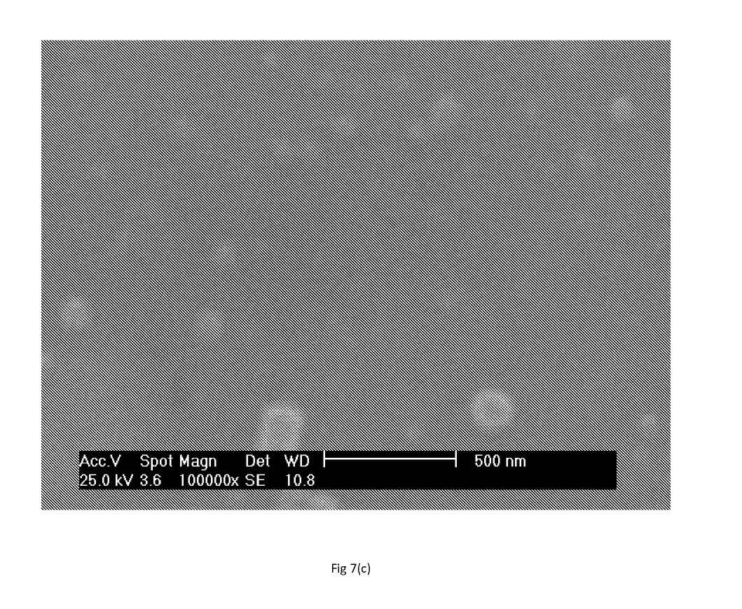

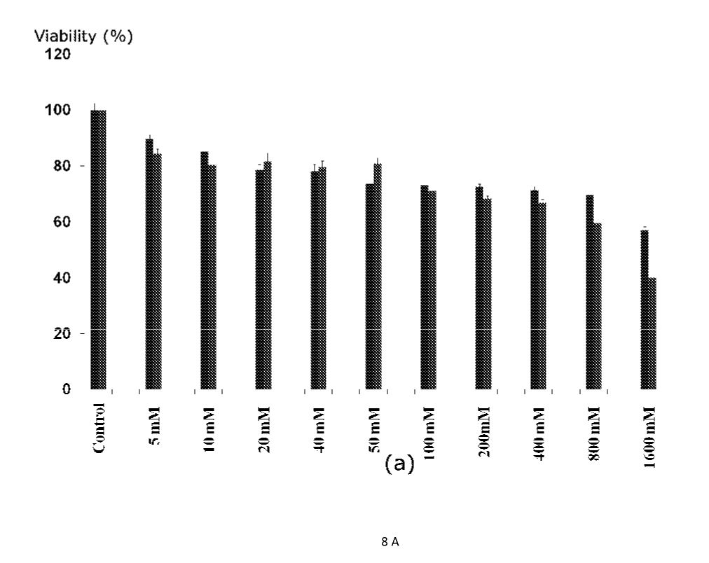

Researchers have investigated the interaction between a coating polymer and Fe₃O₄ particles. For instance, Deng et al. studied polymer interactions in Fe₃O₄/polypyrrole nanocomposites and in Fe₃O₄/polyaniline nanocomposites. They assumed interactions exist between the lone pair electrons of the N atom in the polypyrrole chain or in the polyaniline chain with the 3 d orbital of the Fe atom to form a coordinate bond. Li et al. reported that the interactive mechanism of the oleic molecular adsorbing on the surface of Fe₃O₄ nanoparticles could be due to a hydrogen bond or coordination linkage.²⁷ Zhang et al. reported that Fe₃O₄ nanoparticles could adhere to poly (methacrylic acid) via coordination linkages between the carboxyl groups and iron.²⁸ FIG. 6 shows FT-IR spectra of PEGF coated nanoparticles, before and after cross-linking. The FT-IR spectra of iron oxide exhibit strong bands in the low-frequency region (1000-500 cm⁻¹) due to the iron oxide skeleton. This pattern is consistent with the magnetite (Fe₃O₄) spectrum (band between 570-580 cm⁻¹) or the maghemite (γ-Fe₂O₃) spectrum (broad band 520-610 cm⁻¹). The characteristic band of Fe—O at 572 cm⁻¹ show that the particles consist mainly of Fe₃O₄. On the spectra of macromers the characteristic ester carbonyl stretching bond at 1721 cm⁻¹, asymmetrical C—O—C stretching bond at 1110 cm⁻¹, C—H stretching bond at 2869 cm⁻¹, C═C stretching bond at 1644 cm⁻¹, methylene scissoring and asymmetric peaks at 1454 cm⁻¹ were detected. According to the FTIR spectra, the macromers showed mostly terminal fumarate carboxyl functional groups which are evident by the weakening of the broad —OH end groups absorption at 3500 cm⁻¹. From the cross-linked part of FIG. 6, it is clear that the PEGF has been cross-linked. In addition, SEM study on the cross-linked PEGF coated SPION revealed spherical core shell shape (FIG. 7). As a result we can concluded that PEGF has been cross-linked the magnetic beads and useful particles in order to use in drug delivery has been obtained.

### Biocompatibility of SPION Coated Samples

Primary mouse fibroblasts (L929, adhesive) and human leukemia cells (K562, suspended) from the National Cell Bank of Iran (NCBI), Pasteur Institute, were seeded on glass cover slips in 96 well plates at 10,000 cells per well in 150 μl of medium and incubated for 24 hours. Cells were cultured in Dulbecco's modified Eagle's medium (DMEM) supplemented with 10% fetal bovine serum (FBS) at 37° C. in a 5% CO₂ incubator. After the 24 hour incubation period, 40 μl medium containing SPIONs (5, 10, 20, 40, 50, 100, 200, 400, 800 and 1600 mM iron, measured by atomic absorption) was added to the wells and cells were incubated for additional periods ranging from 24-48 hours. Control cells were incubated with the same culture medium without particles. All particle concentrations and controls were each seeded in five separate wells.

Cytotoxicity was assessed using the MTT (3-(4,5-dimethylthiazol-2-yl)-2,5-diphenyltetrazolium bromide) assay, which is a non-radioactive, colorimetric assay. After 24, and 48 hrs of incubation with the cell-SPION samples, 100 μl of MTT (0.5 mg/mL) was added to each well. Following incubation, the medium was removed and formazan crystals were solubilized by incubation for 20 min in 150 μl, of isopropanol. The absorbance of each well, which assesses viable cells, was read at 545 nm on a microplate reader (Stat Fax-2100, AWARENESS, Palm City, USA).

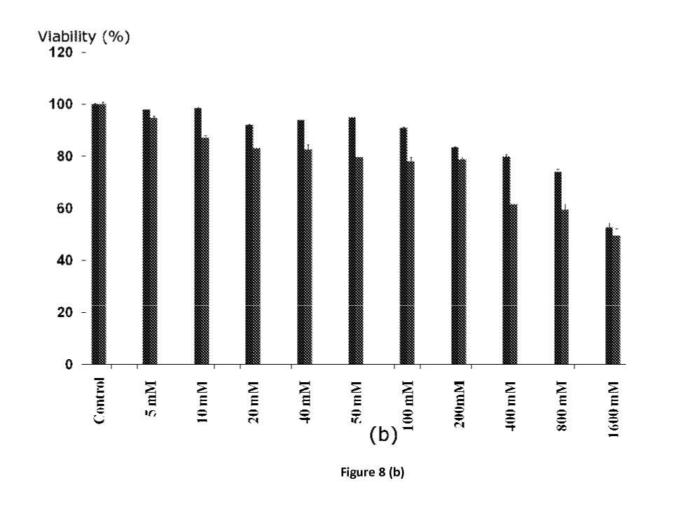

Results of the MTT assays for L929 and K562 cells exposed to all SPION samples are shown in FIGS. 8 (a) and (b), respectively. All synthesized SPION samples demonstrated acceptable levels of cell viability following exposure, with none demonstrating toxic effects at the concentrations tested. In addition the SPION with PEGF coating shows higher biocompatibility in comparison with other coatings such as PVA.

### Drug Release Studies

Tamoxifen (anti-oestrogen drug), which is used to treat breast cancer, was selected as drug. The same amount of non-cross-linked PEGF and cross-linked PEGF (confirmed by atomic absorption) were dried in vacuum. PBS pH 7.4 containing 1 mg drug (TMX) per ml was added to the dried materials and a stable colloidal suspension formed by dispersion of nanoparticles with a homogenizer at 10,000 rpm as well as ultrasonic bath. After 10 h incubation, nanoparticles were collected via centrifugation at 10,000 g and dispersed in fresh PBS. Release data were collected for 300 min. Two milliliter of each sample was centrifuged at selected times and the drug concentration measured in the supernatant by UV spectrometer (Milton Roy Spectronic 601) at 277 nm. The TMX calibration curve was determined from 5-50 μg/ml in PBS pH 7.4. To estimate the amount of drug adsorbed to the surface of the SPION, the concentration of TMX in PBS solution measured before and after interaction with SPION via UV spectrometer. The difference between obtained amounts was suggested as drug uptake by SPION. All release and adsorption measurements were done in triplicates and the standard deviations calculated.

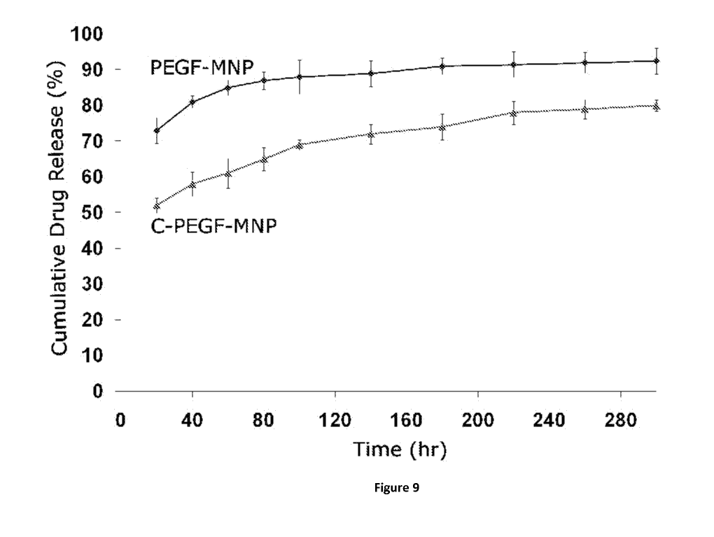

FIG. 9 illustrates drug release from PEGF-MNP (uncrosslinked PEGF magnetic nanoparticles) and C-PEGF-MNP (crosslinked PEGF magnetic nanoparticles) samples. Both the C-PEGF-MNP and PEGF-MNP systems showed burst effects of 52% and 73%, respectively. The cross-linked C-PEGF-MNP is thus, as predicted, able to control the burst effect even in this very simple drug loading system. Better control over burst effect could be reached by drug conjugation with the C-PEGF-MNP.

### EXAMPLE CONCLUSION

Poly(ethylene glycol)fumarate was synthesized from FuCl and polyethylene glycol in the presence of propylene glycol as a new proton scavenger and characterized as a rigid coating. The obtained PEGF has been successfully coated and cross-linked on the surface of SPION. The molarity of base in order to synthesis SPIONs has an influence on the size of obtained magnetic beads. Our results suggest that the compositions based on these unsaturated aliphatic polyesters are great potential in order to develop simultaneous novel carriers for drug and imaging applications.

We claim:

1. A composition comprising: a core comprising of a predetermined amount of superparamagnetic iron oxide nanoparticles;
A shell comprising a predetermined amount of aliphatic unsaturated polyester network, wherein said unsaturated polyester is synthesized in the presence of propylene oxide and wherein said shell contains a predetermined amount of a drug.

2. The composition as claimed in claim 1, wherein said core is made of magnetite.

3. The composition as claimed in claim 1, wherein said aliphatic unsaturated polyester structure is made of a diol and an unsaturated diacid.

4. The composition as claimed in claim 3, wherein said diol comprises of polyethylene glycol, polycaprolactone diol and polyexamethylene carbonate diol.

5. The composition as claimed in claim 3, wherein said diacid comprises of fumaryl chloride and itaconyl chloride.

6. The composition as claimed in claim 3, wherein said unsaturated polyester is crosslinked to the polyester network.

7. The composition as claimed in claim 3, wherein said unsaturated polyester was used without crosslinking.

8. A method for enhancing an image in a medical imaging apparatus, wherein said method comprises of: injecting a composition to a human body, wherein said composition is obtained by:
synthesis of magnetite nanoparticles by a chemical method to obtain a colloidal dispersion of superparamagnetic iron oxide nanoparticles;
adding unsaturated polyester to said colloidal dispersion of superparamagnetic iron oxide nanoparticles;
curing said unsaturated polyester via thermal, redox, or photo initiated crosslinking to obtain said composition, wherein said composition enhances contrast in said medical imaging apparatus.

9. The method as claimed in claim 8, wherein said apparatus is magnetic resonance imaging.

10. The method as claimed in claim 8, wherein said chemical method is co-precipitation, sol-gel, microemulsions, hydrothermal, thermal decomposition, polyol, sonochemical, and electrochemical deposition.

11. The method as claimed in claim 8, wherein said method further comprises combining said composition with a predetermined amount of a drug to obtain a drug loaded composition, wherein said loaded composition is delivered to a predetermined area of a human body and enhances contrast in said medical image apparatus at the same time.

12. The method as claimed in claim 11, wherein said drug is loaded in said composition by adsorption and absorption in said unsaturated polyester and said polyester network.

13. The method as claimed in claim 12, wherein said drug is absorbed in said unsaturated polyester and said polyester network by equilibration of said dried composition in a drug solution.

14. The method as claimed in claim 13, wherein said drug loaded composition release said drug by molecular diffusion through said unsaturated polyester and said polyester network.

15. The method as claimed in claim 12, wherein all of said drug releases in 300 hrs.

16. The method as claimed in claim 15, wherein 73% of said drug is released in first 20 hrs from said unsaturated polyester shell.

17. The method as claimed in claim 15, wherein 52% of said drug is released in first 20 hrs for said polyester network shell.
